(19) United States
(12) Patent Application Publication
(10) Pub. No.: US 2004/0167626 A1
(43) Pub. Date: Aug. 26, 2004

(54) Expandable artificial disc prosthesis

(76) Inventors: Perry A. Geremakis, Manalapan, NJ (US); Roy Hori, Bellevue, WA (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/763,575

(22) Filed: Jan. 22, 2004

Related U.S. Application Data
(60) Provisional application No. 60/441,987, filed on Jan. 23, 2003.

(51) Int. Cl. A61F 2/44

(52) U.S. Cl. 623/17.15; 623/17.14

(57) ABSTRACT

The present invention relates to a spinal implant that allows restoration of movement to an effected vertebral joint. The spinal implant of the current invention also allows the adjustment of the height of the implant so that it can be best fit into the affected joint of an individual patient. One embodiment of the current invention provides an implant having two endplates separated from each other and a central adjuster between the two endplates, wherein the central adjuster is configured to adjust the height of the implant and to articulate with at least one of the two endplates.

### CROSS REFERENCE TO RELATED APPLICATIONS

This application claims priority from provisional application U.S. Serial No. 60/441,987, filed on Jan. 23, 2003.

### BACKGROUND

1. Field of the Invention

This invention relates to devices that would be implanted in joints to allow restoration of motion to effected joint, as well as restoration of the spacing of the effected joint. Specifically, this invention relates to a spinal implant for use in one or more intervertebral disc spaces. The implant will restore motion to the effected functional spinal unit as well as restoring the disc height which will relieve pressure on nearby nerves.

2. Description of Related Art

The spine is composed of a series of vertebrae. Each vertebrae is separated from one another by an intervertebral disc. These discs serve not only as a cushion between the vertebrae, but are also integral to the flexibility of the spine. The disc is composed of a central nucleus surrounded by a series of annular layers, called the annulus. The posterior aspect of vertebrae contains protrusions that form an arch, which surrounds the spinal cord, as well as facets. In addition to the joint formed between the two adjacent vertebrae and the disc between them, adjacent vertebrae form two facet joints. The facet joints bear a small amount (approximately 20%) of the compressive loads going through the spine, and they are also instrumental in limiting the amount of axial rotation. The height of a disc may decrease as a result of degeneration or injury, and as the height of a disc decreases it will begin to bulge outwards, resulting in more compressive load being transferred to the facet joints. This will lead to low back pain, caused by compression and/or irritation of adjacent nerve roots as well as by compression of the spinal cord.

To relieve pain of degenerative or otherwise damaged joints, surgeons have previously fused, or immobilized, these joints. While this may have reduced or eliminated the pain the patient may have been experiencing at the effected joint, functionality of the fused joint was lost. This may lead to pain and degeneration of joints above and below. Eventually, surgeons have sought to restore functionality to a joint as a means for restoring motion as well as releiving pain. Contemporary joint replacement began with Dr. Charnley's hip prosthesis, and now it is standard practice to perform arthroplasty surgery, instead of arthrodesis surgery, to joints such as the hip, knee, shoulder, wrists, elbow, digits, etc.

The same pattern of treatment will hold true for the spine. The current standard of care is to fuse a functional spinal unit (FSU, defined as two vertebrae and the disc between). Fusion of FSUs have been performed by laying graft material over top of posterior spinal elements, introducing graft material into an evacuated disc space, placing metal or polymer cages into an evacuated disc space, implanting rigid screw and rod systems to immobilize FSUs, or through a combination of the above. With any of these methods, pain is often, though not always, reduced or eliminated. However, the function of the joint is lost as a result of being fused. Since the spine contains multiple vertebrae and spinal discs, fusion at one level will not immobilize the entire spine, however, levels above and below a fused FSU will now be forced to bear an increased amount of stress as forces are distributed throughout the spine. The fusion results in an increased amount of stress on adjacent FSU that may accelerate their deterioration, and eventually result in additional fusions to adjacent FSUs. Thus a downward spiral occurs. There always be situations where spinal surgeons will elect fusion, however, there is a growing consensus toward restoring joint function in the spine to avoid this downward spiral.

Previous efforts to address functional restoration of the spine have focused on both nucleus replacements as well as complete disc replacements. Nucleus replacements replace only the nucleus, leaving the annulus as intact as possible. Complete, or total disc replacements replace the nucleus, as well as a portion, if not all, of the annulus. Generally, nucleus replacements tend to be flexible polymer devices that allow restoration of flexibility through the material properties and not necessarily through articulating components. Other nucleus replacements have focused on flexible polymers injected into the nucleus disc space to restore disc height and function. Nucleus replacements are more likely to be used in a minimally invasive surgical approach to the spine, whereas total disc replacements will have a more open approach. Total disc replacements tend to be multi-piece constructs, often made from metal, ceramic, rigid polymers, or a combination thereof, that articulate against their components.

Total disc replacements typically have means to attach, and encourage fusion to, the adjacent vertebral endplates, and an articulation means between these two endplates. There may be one or two articulating surfaces. The central portion between the endplates may be either rigid or act as a cushion in distributing axial compressive loads. In addition, these implants come in a select number of sizes that will require the surgeon to chose the closet size (height) implant for restoration of the natural disc height. However, if the surgeon is not able to chose the implant size that results in appropriate restoration of disc height, functionality may be only partially restored. In addition, if an implant is undersized, the vertebral endplate contacting portions may not properly fuse to these vertebral endplate, which could result in a failed surgery eventually requiring re-operation. If an implant is oversized for the effected disc space, too much compressive load would now be transferred through the implant, and not enough through the facet joints, thereby resulting in abnormal functionality.

There is a need for a spinal implant system that will allow the surgeon to restore functionality to at least one effected FSU. This will include not only restoration of flexibility to the effected joint, but also restoration of the proper disc space height.

### SUMMARY OF THE INVENTION

Described briefly, a preferred embodiment of this invention is to provide a spinal implant that will allow restoration of movement to an effected vertebral level. It is also preferred that such an implant will allow the surgeon to restore the height of the effected disc space to a level determined by the surgeon once the implant has been placed into the disc space. The implant has a first element having a first surface for contacting a first vertebral endplate surface and a second element having a second surface for contacting a second vertebral endplate surface. Said first element has a first articulating surface opposite said first endplate contacting surface. Said first and second surfaces have means for engaging and locking into first and second vertebral endplate surfaces. In addition, said first and second surfaces have means for encouraging fusion into first and second vertebral endplate surfaces, thereby locking first and second implant surfaces to first and second vertebral endplate surfaces. The implant also has internal articulation means positioned between said first and second implant surfaces. Said internal articulation means describe means that allow articulation between said first and second elements. Articulation may be in a manner that will allow restoration any combination of natural disc movements such as flexion, extension, lateral bending, rotation, and translation. The implant also has a third element, having a first articulating surface and means to engage said second element. First articulating surface of said first element can articulate against first articulating surface of said third element. Engagement means of said first and third elements allow for restoration of the disc height once the implant has been placed into the effected disc space.

In one embodiment, the engagement means is a thread, wherein said first element has a thread for engaging a mating thread on second third element. A fourth element would be added to lock the two engaging elements. Said fourth element being a locking screw which would thread against the threaded engagement of first and third elements, and generally perpendicular to this engagement. Alternatively, said locking screws could also thread into the threaded engagement of the first and third elements, as well as locking itself against the outer surface of the engagement elements. Alternatively, a threaded nut could be threaded down along the threading of the third element and against a shoulder on the first element. This type of design would prevent compression, or loss of height, of the device, and would rely on the compressive forces applied to the device once implanted, to prevent further distraction of the device. Other locking means, such as a press fit pin or adhesives, could be applied to lock the engagement elements together once the disc height has been appropriately restored by the implant.

Another embodiment would have the above mentioned first, second, third, and fourth elements, but instead of the first and second elements articulating freely, and unconstrained, the articulating surface of one of the two elements could be preassembled, or captured, at least in part, by the other element. This would result in an assembly that the surgeon could implant at once, instead of in multiple pieces, thereby decreasing the time required for surgery.

A preferred embodiment of this invention is a surgical method of use for restoring the flexibility of an FSU as well as restoring the height of said FSU. This method involves the steps of approaching the disc space from either an anterior, anterolateral, lateral, posterior, or transforaminal direction; removing necessary disc material; preparing the vertebral endplates to accept the vertebral endplate contacting surfaces of the implant, through rasping, chiseling, broaching, or other acceptable means to expose an appropriate amount of bleeding endplate bone and contour the vertebral endplates, using either manual or powered instruments (such as ossicilating or reciprocating saws, pneumatic coblation techniques, ultrasonics, lasers, etc.); using implant trial gauge(s) to asses the appropriateness of the endplate preparation in terms of conformance to the vertebral endplate contacting surfaces of the implant, depth of the channel or cavity created by the endplate preparation, uniformity of the channel or cavity created by the endplate preparation, degree of bleeding bone, and/or strength of the exposed bone that will support the vertebral endplate contacting surfaces of the implant; inserting the implant into the disc space, expanding the implant to restore the disc height of the effected FSU, applying means to lock the height of the implant; close wound. Other variations of the surgical method are also contemplated and will be described.

By having an implant that can expand, or that the surgeon can expand, to restore the appropriate amount of disc space height, greater flexibility is provided to the surgeon, and there exists the potential for a single implant accommodating all disc levels, thereby reducing potential for confusion, mislabeling of implants, etc.

### BRIEF DESCRIPTION OF THE DRAWINGS

Preferred embodiment(s) of the present disclosure are described herein with reference to the drawings wherein:

FIG. 1 is an isometric view of an artificial disc prosthesis in accordance to the principles of the present invention.

FIG. 2 is a side view schematically of the artificial disc prosthesis of FIG. 1.

FIG. 3 is an isometric view of another embodiment of the artificial disc prosthesis in accordance to the principles of the present invention.

FIG. 4 is an isometric view schematically of an endplate of the artificial disc prosthesis of FIG. 3.

FIG. 5 is an isometric view of a further embodiment of the artificial disc prosthesis in accordance to the principles of the present invention.

FIG. 6A is a side view schematically of the artificial disc prosthesis of FIG. 5.

FIG. 6B is a front view schematically of the artificial disc prosthesis of FIG. 5.

FIG. 6C is a top view schematically of the artificial disc prosthesis of FIG. 5.

FIG. 6D is an isometric view schematically of the artificial disc prosthesis of FIG. 5.

FIG. 7 is an illustration of the groove patterns and hole patterns on the endplate of the artificial disc prosthesis of FIG. 5.

FIG. 8 is an isometric view of another embodiment of the artificial disc prosthesis in accordance to the principles of the present invention.

FIG. 9A is a side view schematically of the artificial disc prosthesis of FIG. 8.

FIG. 9B is a front view schematically of the artificial disc prosthesis of FIG. 8.

FIG. 9C is a top view schematically of the artificial disc prosthesis of FIG. 8.

FIG. 9D is an isometric view schematically of the artificial disc prosthesis of FIG. 8.

FIG. 10 is an isometric view of another embodiment of the artificial disc prosthesis in accordance to the principles of the present invention.

FIG. 11A is a side view schematically of the artificial disc prosthesis of FIG. 10.

FIG. 11B is a front view schematically of the artificial disc prosthesis of FIG. 10.

FIG. 11C is a top view schematically of the artificial disc prosthesis of FIG. 10.

FIG. 11D is an isometric view schematically of the artificial disc prosthesis of FIG. 10.

FIG. 12 is an isometric view of another embodiment of the artificial disc prosthesis in accordance to the principles of the present invention.

FIG. 13A is a side view schematically of the artificial disc prosthesis of FIG. 12.

FIG. 13B is a front view schematically of the artificial disc prosthesis of FIG. 12.

FIG. 13C is a top view schematically of the artificial disc prosthesis of FIG. 12.

FIG. 13D is an isometric view schematically of the artificial disc prosthesis of FIG. 12.

FIG. 14 is an isometric view of another embodiment of the artificial disc prosthesis in accordance to the principles of the present invention.

FIG. 15A is a side view schematically of the artificial disc prosthesis of FIG. 14.

FIG. 15B is a front view schematically of the artificial disc prosthesis of FIG. 14.

FIG. 15C is a top view schematically of the artificial disc prosthesis of FIG. 14.

FIG. 15D is an isometric view schematically of the artificial disc prosthesis of FIG. 14.

FIG. 16 is an isometric view of another embodiment of the artificial disc prosthesis in accordance to the principles of the present invention.

FIG. 17A is a top view schematically of the artificial disc prosthesis of FIG. 16.

FIG. 17B is an isometric view schematically of the artificial disc prosthesis of FIG. 16.

FIG. 17C is a front view schematically of the artificial disc prosthesis of FIG. 16.

FIG. 17D is a side view schematically of the artificial disc prosthesis of FIG. 16.

FIG. 18 is an isometric view of another embodiment of the artificial disc prosthesis in accordance to the principles of the present invention.

FIG. 19A is a side view schematically of the artificial disc prosthesis of FIG. 18.

FIG. 19B is a front view schematically of the artificial disc prosthesis of FIG. 18.

FIG. 19C is a cross-sectional view schematically of the artificial disc prosthesis across 19C-19C of FIG. 19B.

FIG. 19D is an isometric view schematically of the artificial disc prosthesis of FIG. 18.

### DETAILED DESCRIPTION OF THE INVENTION

Implant 1:

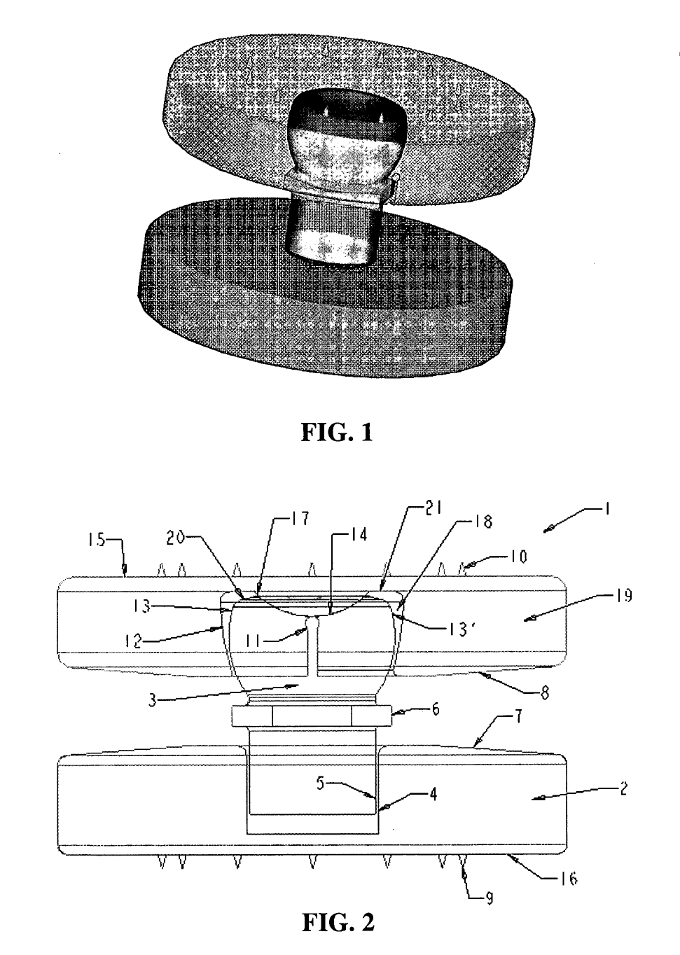

Implant 1 is composed of three elements: upper endplate 19, lower endplate 2, and central articulating post 3, as shown in FIGS. 1 and 2. Upper endplate 19 has protrusions 10 on the top surface 15, top surface being configured to engage the lower vertebral endplate of the upper vertebrae of the FSU. Lower endplate 2 has protrusions 9 on surface 16, said surface 16 configured to engage the upper vertebral endplate of the lower vertebrae FSU. Protrusions 9, 10 are shown to be spikes, however, these can be any type of surface roughenings, such as teeth, ridges, posts, porous coated (beaded) surfaces etc., that can engage and lock the Upper and Lower Endplates into the respective vertebral endplates. There can be a single protrusion, or multiple ones. Additionally, surfaces 15 and 16 can be treated or coated with substances to promote bony fusion of the vertebral endplates into and around the surfaces 15 and 16.

Central articulating post 3 is matingly engaged to lower endplate 2, in the preferred embodiment, through threaded surfaces 4 and 5. The opposite side of post 3 articulates against the upper endplate 19. Surface 17 of upper endplate 19 articulates against surface 14 of the post 3. These articulating surfaces are configured to allow axial rotation, translation, as well as bending (mimicking flexion/extension bending as well as lateral bending of the disc). Alternatively, the articulating surfaces may be convex (13)-concave (14)-convex (13′), instead of one continuous surface (14), on either the central post, or one of the articulating endplates. Surfaces 20 and 21 serve to act as stops against over flexion/extension, and/or lateral bending can occur. Furthermore, the articulating surfaces can be configured to have any combination of axial rotation, translation, bending (mimicking flexion/extension, as well as lateral bending).

Preferably Implant 1 is pre-assembled prior to implantation, and is implanted as a single construct. This is accomplished by inserting the articulating end of the post 3 into the cavity 18 of the upper endplate 19. Resilient deformation of the wall surrounding cavity 18, through elastic expansion and contraction of slot 11, allows the post 3 to be “snapped” in place. Once the articulating head of the post 3 is engaged within cavity 18, casual handling of the implant assembly will not cause separation of the components, thereby allowing implantation into the disc space as a single construct, and not have to have the implant assembled within the disc space. There may be multiple slots to accommodate various specific designs, or simply as single slot as shown in Implant 1.

The implant is inserted into the evacuated vertebral disc space in a compressed state. Once it has been appropriately positioned, the surgeon will expand the implant so that surfaces 15 and 16 bear against the respective, prepared vertebral endplates. Protrusions 9 and 10 will penetrate through the vertebral endplates and into the vertebral bodies. The portions of surfaces 15 and 16 that do not contain protrusions 9 and 10, will bear against the respective vertebral endplates. Expansion of the implant will continue until the surgeon deems that an appropriate amount of tension has been restored to the FSU. For Implant 1, expansion is achieved by rotation of central post 3, through the hex-nut shaped extension 6. The surgeon can use a tool with an end that will mate with the shape of 6, to allow rotation of nut 6 and therefore post 3 with respect to lower endplate 2. Once this overall height has been restored by the expanded implant, the surgeon may elect to lock the post 3 to the lower endplate, thereby preventing further expansion or contraction of the implant once the surgery is completed. Locking can be accomplished in several ways, many of which will be described below. For instance with Implant 1, an additional threaded nut (not shown) can be threaded down against surface 7 of lower endplate 2, thereby preventing contraction of the implant; the more or less constant compressive loading that goes through the spine on a daily basis will ensure that additional expansion of the implant will not continue. However, the additional threaded nut could contain means (surface roughenings) to dig into surface 7, frictionally locking with surface 7. Additionally, other means such as adhesives, spiking/swaging, etc. can be used.

For example, and not limitation, all the implants and instruments described herein, will be made from materials appropriate for surgical use; preferably articulating surfaces will be metals (stainless steels, cobalt-chrome alloys, titanium), polymers, or ceramics, or a combination thereof. Although described as having the articulating surface in upper endplate 19, this can be reversed, and have the upper endplate 1 contain the non-articulating threaded surface to engage the central post 3.

The preferred method of use will now be described. The surgeon will determine the most appropriate approach to the spinal disc space, whether it be anterior, anterolateral, lateral, posterior, posterolater, transforaminal. Once the disc space has been reached, the necessary disc material is removed, using standard surgical instruments, such as scalpels, curettes, rongeurs, etc. Not all of the disc material need be removed, but generally, all of the nucleus will be, and depending on the surgical approach to the disc space, a portion of the annulus at the approach vector to the disc space will also be removed.

Distraction of the disc space may be performed if necessary to achieve the desired surgical results, and this can be performed through paddle type distractors (ones that are inserted with their small profile, then rotated 90 degrees to distract the disc space, similar to the action of a cam). Alternatively, distractors such as lamina spreaders, scissors-type distractors that engage the vertebral bodies, scissors-type distractors that do not penetrate into the bone of the vertebrae, but rather distract off of the vertebral endplates. Screws may be inserted into portions of the vertebral bodies, either temporarily, or permanently, and distraction may be performed off of these screws. Said screws for distraction may be placed in the anterior portion of the vertebral bodies (envisioned two screws per FSU), or in the lateral portion of the vertebral bodies (envisioned up to four screws per FSU, with two screws on each side of the vertebral body). The distraction may be maintained throughout the surgery, or can be used for selected portions, such as endplate preparation.

The endplate preparation instruments, be it standard surgical instruments such as chisels, box chisels, rasps, etc., are used to form the vertebral endplates to best accept the vertebral endplate contacting surfaces of the implant to be inserted.

A trial spacer is then used to assess the disc space for an appropriately sized implant. Although the implant is an expandable, and therefore height adjustable one, there may be more than one size implant offered within the surgical kit. As a comparison, a traditional implant may have height offerings in one or two millimeter increments, and have five to ten heights. An implant that is height adjustable could be such to accommodate all necessary heights, or there may be two or three expandable implants per surgical kit, which will accommodate all necessary disc heights. Depending on the number of implants within the surgical kit, a similar number of trial implant would be offered. If the surgeon is unsatisfied with the result of the trialing, additional endplate preparation may be performed, and this iterative process is continued until the desired results are achieved.

Once the final trialing has been completed, the surgeon will introduce the expandable implant in a contracted state, orient it within the disc space as necessary, fit it into at least one of the prepared vertebral endplate surface/cavity/channel, and then expand the implant. During expansion of the implant, the surgeon will ensure that both vertebral endplate contacting surfaces of the implant properly engage the prepared vertebral endplates. Once the implant has been expanded to the height as determined by the surgeon, for example to achieve the necessary distraction, stability, and restoration of the “tension-band” effect, the surgeon will ensure that the appropriate kinematics (motion) have been restored to the effected joint. Once satisfied that the is appropriated sized, and functional, it is then locked in place. This is described in more detail below, and varies from implant to implant, but generally, a jam-screw or jam-nut is used to prevent contraction of the implant once it has been inserted and locked into place. Closure is performed in accordance with standard surgical practices.

Other variation of this technique will be described below, for example, using fusion promoting materials to enhance fixation of the vertebral endplate contacting surfaces of the implant to the vertebral endplates themselves. Additionally, the implants described below may be used in multiple vertebral levels.

Implant 30:

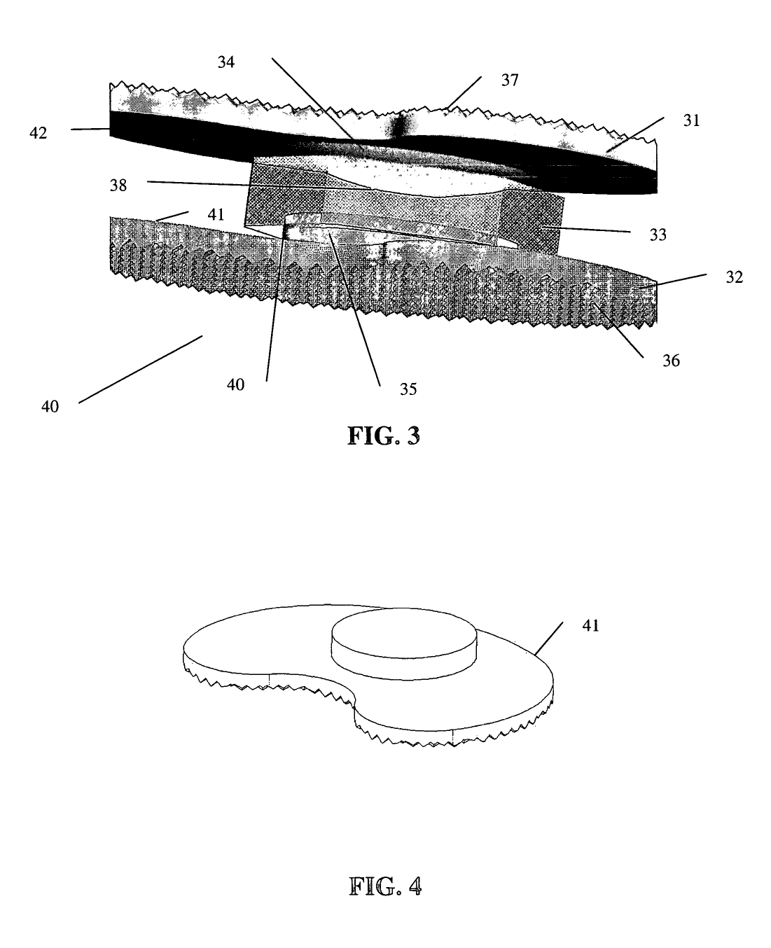

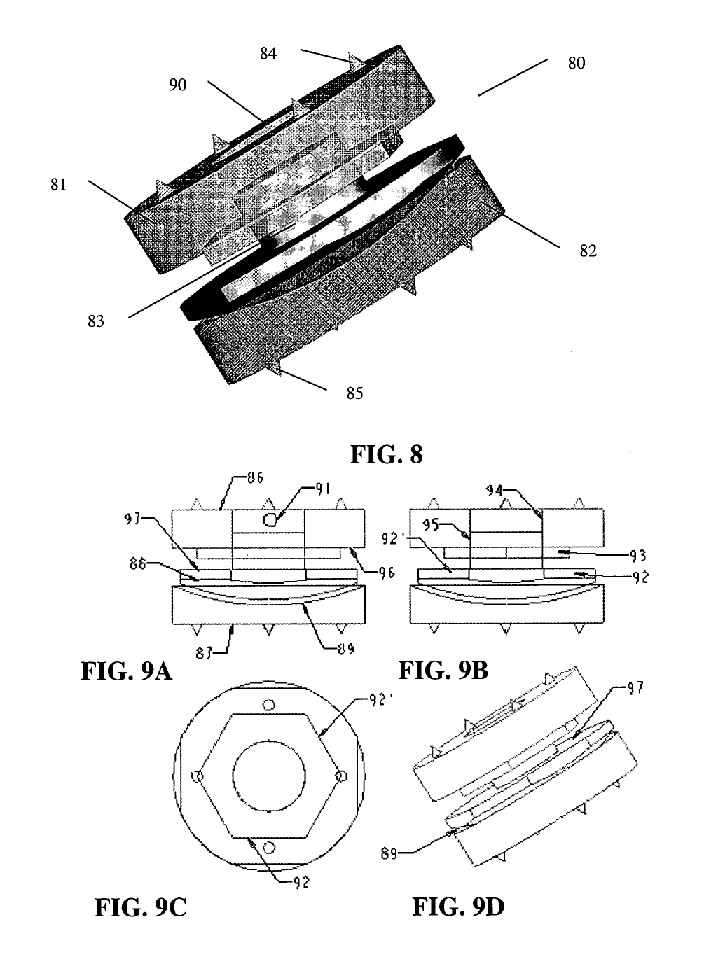

Implant 30, as shown in FIG. 3, is composed of three elements, upper endplate 31, lower endplate 32 and central post 33. Upper and lower endplates 31 and 32 engage, respectively, the lower endplate of the upper vertebrae and the upper vertebral endplate of the lower vertebrae, of the disc space into which the implant is inserted. Surface roughenings 36 and 37 help to penetrate into the vertebral endplates thereby locking the endplates 31 and 32 into their adjacent vertebral endplates. As described above, alternative surface roughenings are contemplated. The axial profile 41 of the upper and lower endplates need not be circular, as shown in Implant 1, but can be kidney shaped as shown in Implant 30 (FIG. 4). Alternatively, this profile can be oval, “figure 8-style”, or any other shape that can best be used to ensure the successful implantation of the artificial disc.

Articulation of Implant 30 is accomplished through movement between surfaces 34 and 38. Although shown as a constant radius of convexity and concavity, respectively, these surfaces can be composed of multiple radius, flats, or the combination thereof. Additionally, surfaces 34 and 38 can be composed of convex and concave surfaces that blend into each other on one of the component elements, to form a wavy curve. For example, articulating surface 34 of the upper endplate can have a convex outer region, transitioning into a concave central region, similar to the surfaces 14 and 17 of Implant 1. These articulating surfaces are configured to allow axial rotation, translation, as well as bending (mimicking flexion/extension bending as well as lateral bending of the disc). Surfaces 41 and 42 act as positive stops thereby limiting the degree to which flexion/extension and lateral bending can occur. Alternatively, the central post 33 may be dimensioned to flexion/extension and lateral bending to only stop once the upper and lower endplates contact each other.

Expansion of the implant is performed in a manner similar to that described in Implant 1. Rotation of the central post 33 against the lower endplate 32, specifically, surface 40 engaging with surface 35. Preferably, surfaces 35 and 40 are threadably engaged, and this threading allows for rotation, and therefore expansion of the implant to occur. Although central post 33 is shown with an exterior hex profile to allow mating engaging with an expansion tool (not shown, but contemplated to be a wrench, a torque measuring wrench, or a torque limiting wrench), other means of engagement to an expansion tool is contemplated. For example, multiple holes can be applied to the exterior surface of 33, for engagement with a “tommy-bar”, ratchet, or other appropriate means, hereafter referred to in this application as “tommy-bar”, allowing the surgeon to rotate the central post and adjust the height of the implant accordingly. These holes for engagement with a “tommy-bar” may be threaded. Such a threaded rod could not only be used to expand the implant, but also insert the implant into the prepared disc space.

Central post 33 can be designed to capture protrusion 35 of lower endplate 32, not only through the threaded surfaces, but a lip could be added to the top of protrusion 35 as well as a lip to the lower surface of 33, such that these respective lips would overlap and prevent further expansion of the implant.

Similar means to lock the expanded height of the implant are contemplated, as well as applying at least one jam-screw (not shown), into and through aforementioned “tommy-bar” engaging holes (not shown), so that the jam-screw will abut and frictionally engage the threaded surface of protrusion 35 of lower endplate 32. Alternatively, threaded surface 35 can have a series of holes drilled into 35, in a upwardly spiraling helix, so that the implant height can be locked into place at any one of several intervals, as determined by the spacing of the series of helical holes.

Implant 50

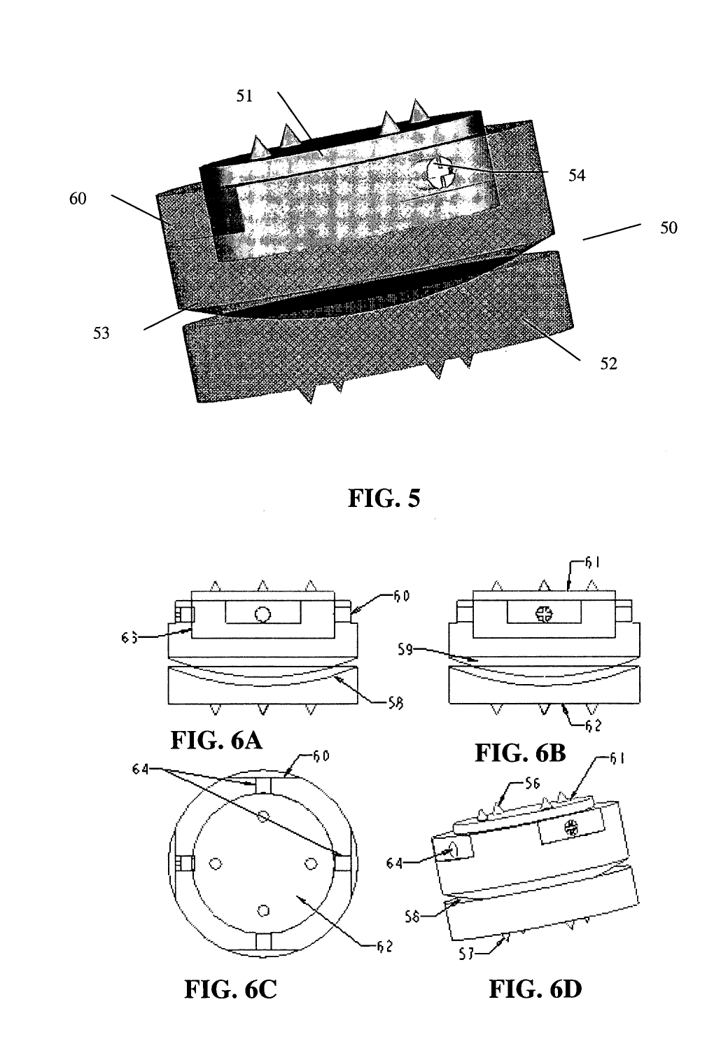

Implant 50, as shown in FIGS. 5-6D, is a three piece construct, held together in part by one or more jam screws 54. The implant is composed of an upper endplate 51, a lower endplate 52, and a central portion 53. The endplates engage the vertebral bodies in a manner similar to that described previously, through protrusion 56 and 57, and endplate contacting surfaces 61 and 62. Once implanted, the surgeon can expand the implant by rotation of the central portion 53. Preferably, surfaces 51 and 63 are threaded to engage each other. As central portion 53 is rotated against upper endplate 51, upper endplate 51 is forced away from the lower endplate 52, which results in an overall increase in height of the implant 50. A series of holes 64 can be used to turn the central portion 53, through the use of the aforementioned “tommy-bar” or a threaded rod. Alternatively, an instrument can rotate 53 by engaging flats 60, or an instrument can rotate 53 through a combination of engaging at least two flat surfaces 60 as well as at least one of the holes 64.

Articulation, as described previously, occurs through contact of surfaces 58 and 59. Similar variation may be considered, as described previously in Implant 1. For instance, the radius of 59 can be smaller than that of 58, thereby allowing translation to occur between the two surfaces.

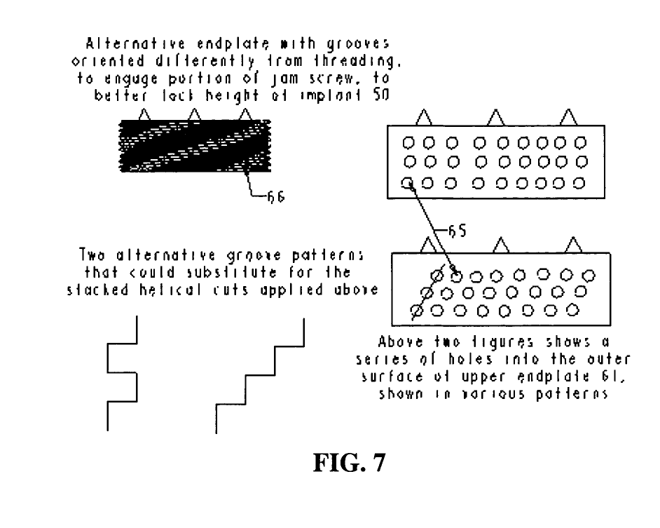

The overall expanded height of the implant is set, or locked, by applying at least one jam screw 54 through at least one hole 64 so that they abut and slightly deform into the surface 51. As described previously for Implant 30, and as shown in FIG. 7, surface 51 may contain a series of holes 65 to accept a portion of the jam screws. Alternatively, there may be a series of grooves 66 along a part of surface 51, in a pattern different from that of the threading, allowing a portion of the jam screw to be placed into said groove to lock the height. The different pattern of the grooves may have a different pitch, be of a stepped pattern, etc. Additionally, said hole 65 may also be in a series of patterns just described (helical, stepped, etc.). Alternatively, 54 can be rivets, pins, expandable plugs (made from shape memory alloys).

Implant 80

Implant 80, as shown in FIGS. 8-9D, has the following components: an upper endplate 81, a lower endplate 82, a central portion 83, and a threaded jam nut 93. Inner surface 94 of upper endplate 81 is threaded to receive the threaded outer surface 95 of central portion 83. This allows for expansion of the overall height of the implant through rotation. Rotation can be accomplished through means previously described for Implant 30, although in implant 80, it is shown through a wrench type instrument engaging two or more flats on central portion 83, for example flats 92 and 92′.

Once the implant has been expanded to the desired height, a jam nut 93 is threaded against surface 96 of the upper endplate. The nut is jammed and locked into place via friction lock. The engaging surface of jam nut 93 can be roughened to better frictionally engage and lock into surface 96. Additionally, a counter torque can be applied simultaneously to upper endplate 81 to increase the amount of friction lock. Another embodiment would allow the jam nut to be initially countersunk into surface 97 of central portion 93.

Articulation occurs through movement of surface 89 against 88. Previous variations of articulating surfaces of Implants 1 and 30 can be applied to implant 80.

Once the implant 80 has been locked into a final height, additional graft material (autograft, allograft, DBM, calcium phosphate, etc.), fusion extenders, bone growth proteins, collagen, bone composites, etc., or a combination of these may be introduced into cavity 90, through hole 91. A screw or end cap (both not shown) may be applied to seal the opening of the hole 91, and prevent said fusion promoting material from backing out of the cavity 90. This additional material will help fusion of the implant to the endplate contacting surface 86 of upper endplate 81. Additionally, as the bone fuses into cavity 90, a macro-lock occurs, a similar phenomenon occurs when bone grows up to and around threaded surfaces 94. Alternatively, vertebral endplate contacting surfaces can have grooves, chanels, chambers, recesses etc., that will allow bone material to grow into and lock the vertebral contacting endplate surfaces of the implant, to the vertebral endplates.

A further embodiment would have the implant endplates have means to thread (not shown) into the vertebral endplates. This could be in the form of threading on a single post, or threading on the external surface defining the circumference of the implant endplates.

Implant 100:

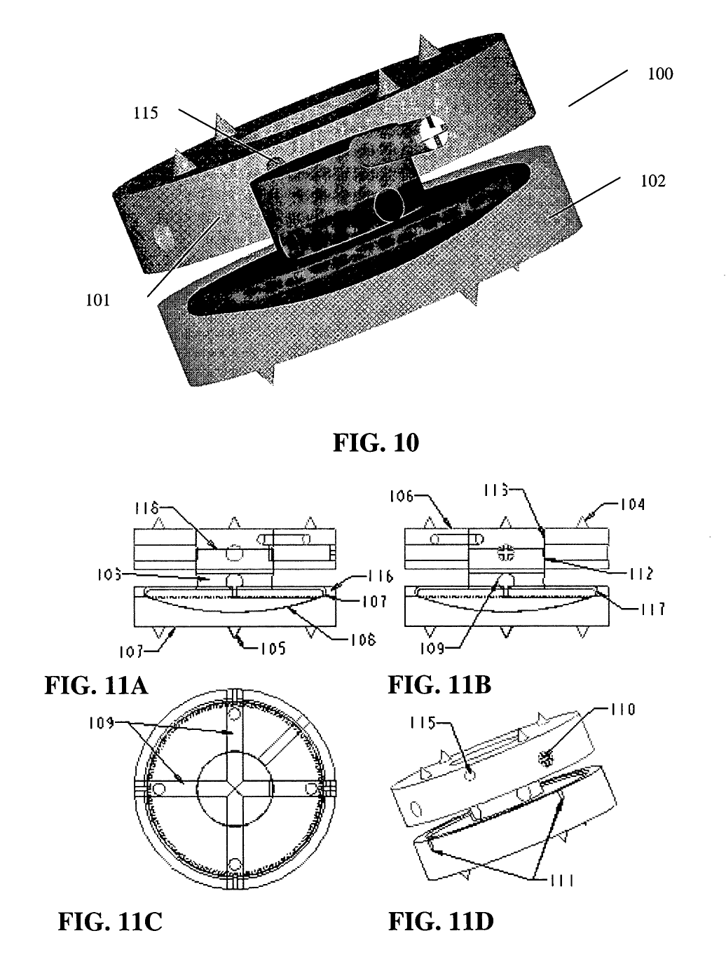

Implant 100, as shown in FIGS. 10-11D has the following elements: upper endplate 101, lower endplate 102, and central portion 103. Implant 100 expands its height through rotation of 103 against 101, specifically, threaded surfaces 112 and 113. As 103 is rotated, 101 expands further away from 103 and 102, thereby increasing the overall height of the implant, restoring the desired disc height. A series of jam screws 110 can be placed through holes 109 so that they abut and frictionally engage a part of threaded surface 112 of the central portion 103. Alternative means of engagement of screws 110 to central portion 103, as described previously in implant 80 can also be used (grooves in threads, a series of holes in 103, etc.).

Implant 100 can be preassembled prior to implantation similar to Implant 1. The articulating head 116 of central portion 103 can be “snapped” into cavity 117 of lower endplate 102. Elastic deformation through expansion of slots 111 of endplate 102 will facilitate this. Once assembled, the lip 116 will overlap the outer articulating boundary of 107, keeping 102 captured. Also, 116 will serve to act as a stop against motion (translation, lateral bending, and flexion/extension). Articulation occurs between surfaces 107 and 108.

As described with implant 80, introduction of fusion promoting material can be done through hole 115 and into cavity 114. Therefore, as endplate engaging surface 106 bears against the vertebral endplate, virtually no load is transferred through the inserted fusion promoting material in cavity 90, initially. Over time, as this material becomes, or promotes growth of bone, load will be borne by surface 106 as well as surface 118 of the central portion 103.

Implant 130:

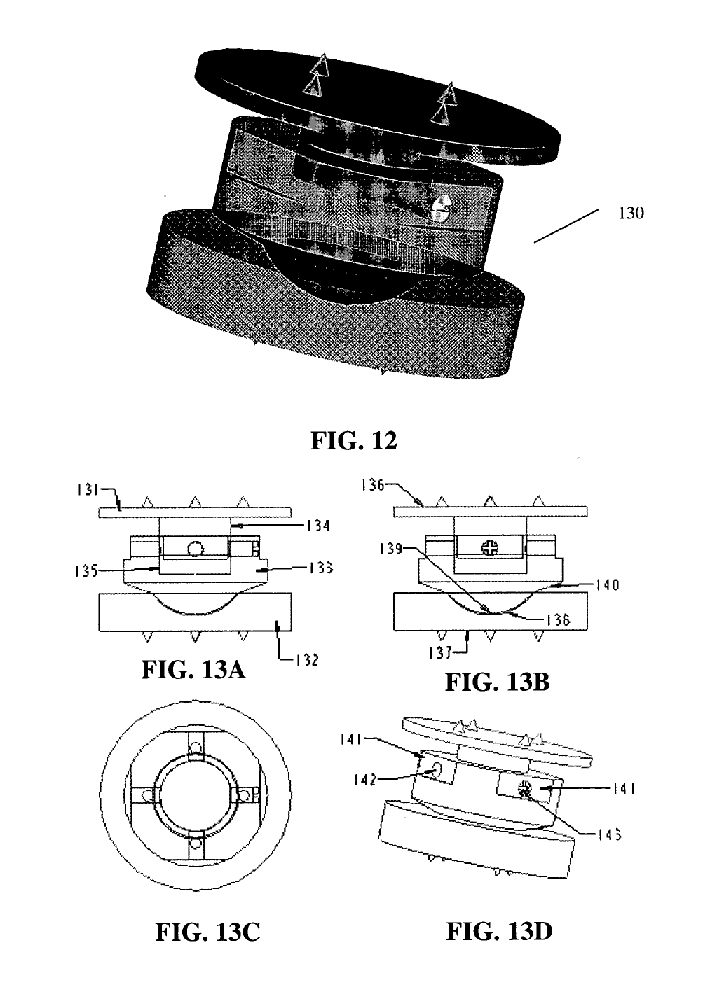

Implant 130, as shown in FIGS. 12-13D, is composed of elements: upper endplate 131, lower endplate 132, and central portion 133. Implant 130 is similar to implant 30. Expansion is done through rotation of the central portion 133 against 134, specifically the threaded portions 135 and 134 of each, respectively. Once the desired height is determined, a series of jam screws 143 can be inserted through holes 142, to frictionally engage surface 134 of upper endplate 131. Other means of engaging jam screws to upper endplate 131, as previously described in implant 50 can also be applied to implant 130. Rotation can be done through engagement of a “tommy-bar” or a threaded rod into holes 142, engagement of a wrench-type instrument against surface 141, or a combination of these methods.

Articulation occurs between surfaces 138 and 139. Note the surface 140 acts as a positive stop against excessive lateral bending as well as flexion/extension.

Implant 200:

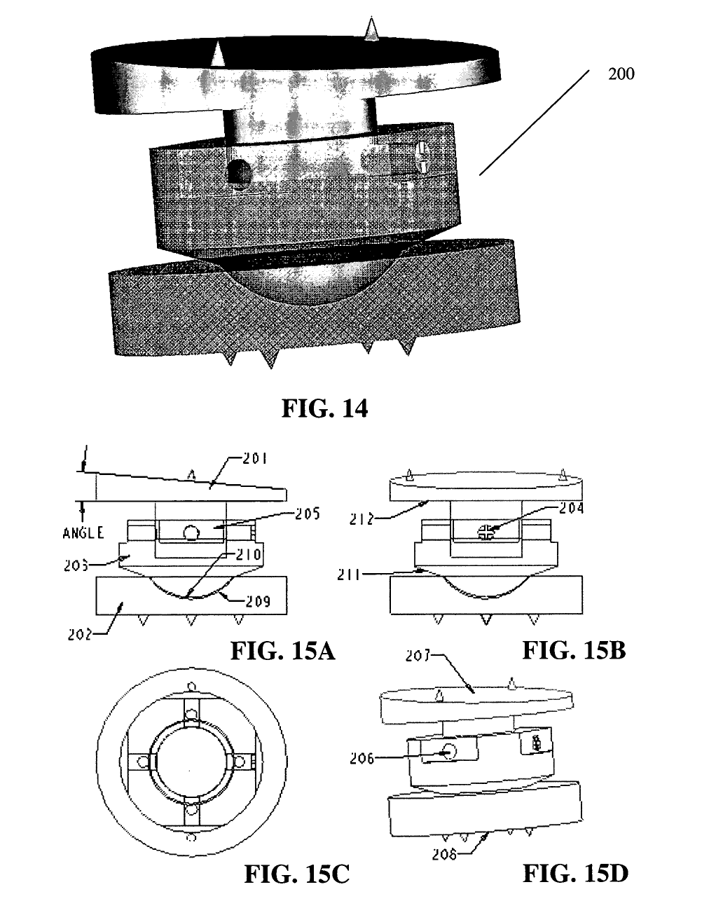

Implant 200, as shown in FIGS. 14-15D, is composed of an upper endplate 201, lower endplate 202, a central articulating portion 203. This implant is very similar to implant 130 in terms of function. However, the upper vertebral engaging surface 207 of upper endplate 201 is angled (θ) with respect to surface 212. Angulation can applied to either endplates, to best restore the original geometry of the disc space of which the implant will be inserted. Lordosis, kyphosis, scoliosis, or combination of these, can be restored or corrected, depending on the angle and orientation of said angle chosen.

Implant 230:

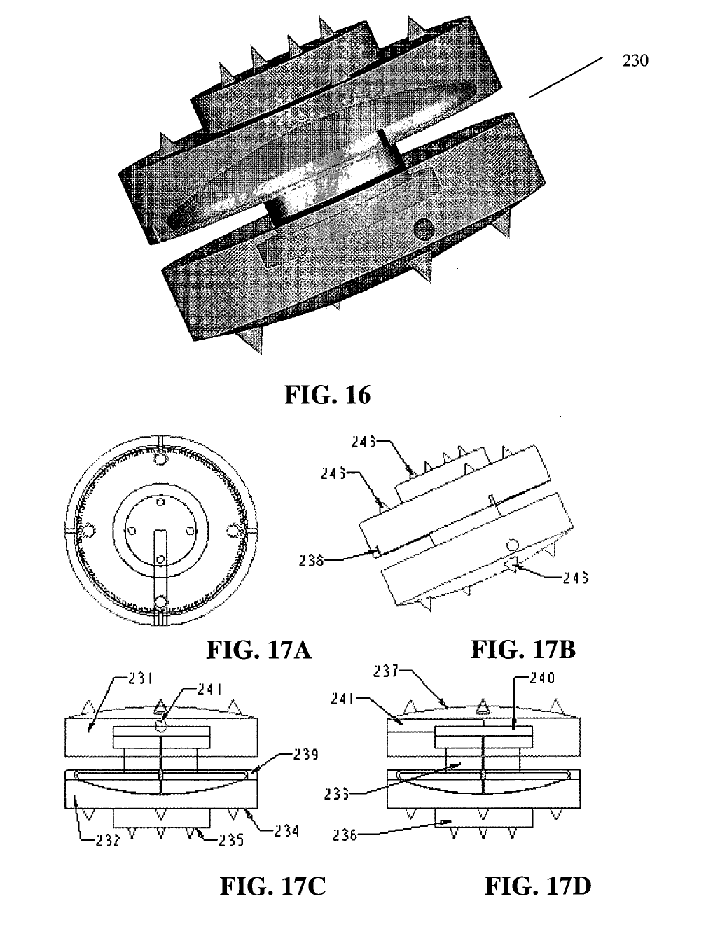

Implant 230, as shown in FIGS. 16-17D, is composed of three elements: upper endplate 231, lower endplate 232, and central articulating portion 233. Articulation is accomplished through means described in implant 100. The implant 230 can be inserted as a single unit, as described for Implant 1. Implant 230 shows variations of vertebral endplate contacting surfaces 237 from upper endplate 231 and surfaces 234 and 235 on lower endplate 232. Surface 237 is convex, and although it is shown with a constant radius, it is envisioned that surface 237 can have varying radii, yet still be generally convex. Alternatively, only a portion of surface 237 need be convex, for example, the outer rim may be flat, yet the central portion can be convex. Such a flat outer rim surface is shown by 234 of the lower endplate 232. Lower endplate 232 also shows a stepped vertebral endplate contacting surface. This would correspond with a vertebral endplate machined or contoured to accept such a stepped vertebral endplate contacting surface. Protrusions 243 on vertebral endplate contacting surfaces still remain, and as described previously, can be of a variety of suitable types.

Trial:

A trial is composed of two elements, an upper endplate and a lower endplate. Other elements, such as jam screws, or jam nuts (not shown), may be incorporated to accommodate different functions, such as maintaining the disc height, until the implant is readied to be inserted. Expansion is performed in a manner described previously for aforementioned implants. Trial may comprise two threaded surfaces, which can be rotated to increase of decrease the heights of the Trial. By using a hex-type rotation tool (not shown) to engage at least two of the sides, the Trial is expanded. A second wrench tool (not shown) can also be used to apply a counter torque, said second wrench tool engaging at least one of slots. Instead of slots, holes and rods could be used to rotate and adjust the height of the implant, in a manner described previously. Said second wrench or first wrench can both be applying torques to cause rotation, or one could be applying a torque simply to keep that portion of the trial from moving, while the other wrench causes rotation of the other endplate portion. Instead of slots, holes and rods could be used to rotate and adjust the height of the implant, in a manner described previously.

The trial will have vertebral endplate contacting surfaces, that will closely resemble the corresponding vertebral endplate contacting surfaces of the implant to be inserted. Trial may have two such endplate configurations: one being a convex surface, roughly corresponding to a similarly convex surface of implant 230, as well as to the prepared vertebral endplate itself. The other vertebral endplate contacting surface of trial is a stepped surface, comprising two surfaces separated by a wall/height. Said stepped surfaces also correspond, for example, to surfaces 234 and 235 of implant 230.

A further function of the trial would be to provide the surgeon with a sense of the stability of the system, once the trial has been expanded within the prepared vertebral endplates to the height the surgeon determines will be appropriate for the implant itself.

Implant 360:

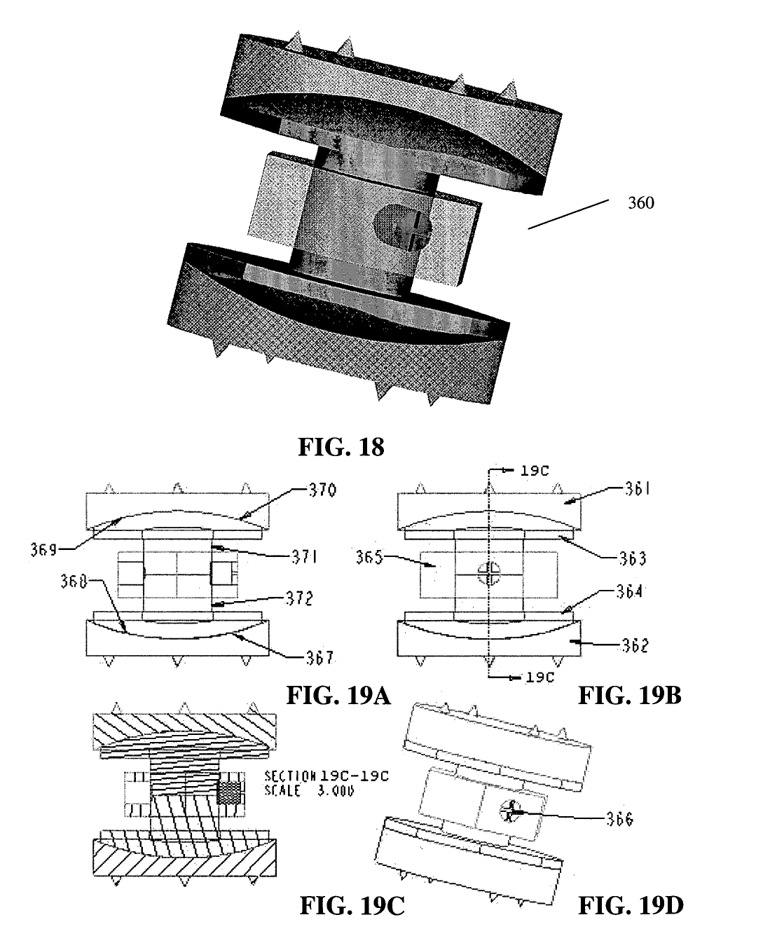

Implant 360, as shown in FIGS. 18-19D, is composed of an upper endplate 361, a lower endplate 362, an upper articulating portion 363, a lower articulating portion 364, a central retaining ring 365, a jam screw 366. Implant 360 differs from the previously described inventions in that implant 360 contains two pairs of articulating surfaces: 367, 368 as well as 369, 370. Surfaces 371 and 372 of the upper and lower articulating portions, respectively, are threaded, each in a direction opposite from the other (left and right threading). Additionally, the retaining ring 365 has internal threading to match, half of the threading is in one direction, the other half is in the other direction, both to match the corresponding threaded portions of the articulating portions. Similar variations to the articulating surfaces, vertebral endplate contacting surfaces of the implants, locking means, etc., that have been described previously in this text, can be applied to implant 360 as well.

It is understood that the inventions and features disclosed herein are not limiting, and that features discussed in one embodiment may easily be applied to others. The ideas described herein can be interchangeable with the various embodiments.

We claim:

1. An artificial intervertebral implant for replacing at least a portion of an intervertebral disc, comprising:
two endplates separated from each other, the two endplates each having an outward facing surface that faces away from each other so that a height of the implant is defined by a separating distance between each of the outward facing surfaces;
a central adjuster between the two endplates and configured to adjust the height of the implant by changing the separating distance between the outward facing surfaces of the two endplates, the central adjuster having at least one articulating surface configured to articulate between the adjuster and at least one of the two endplates.

2. The artificial intervertebral implant of claim 1, wherein the central adjuster is threadingly engaged with a further of the two endplates so that rotating the central adjuster in relation to the further of the two endplates causes a change in the separating distance between the endplates.

3. The artificial intervertebral implant of claim 2, wherein the central adjuster has two flat surfaces between which a matingly engaging tool may be inserted for rotating the central adjuster in relation to the further of the two endplates.

4. The artificial intervertebral implant of claim 2, wherein the central adjuster has at least one cavity into which an engaging tool may be inserted for rotating the central adjuster in relation to the further of the two endplates.

5. The artificial intervertebral implant of claim 4, wherein an inner surface of the cavity is threaded so that the engaging tool may be matingly threaded into the cavity.

6. The artificial intervertebral implant of claim 4, wherein the cavity is configured to be an aperture that penetrates through the central adjuster.

7. The artificial intervertebral implant of claim 1, further comprising a lock which is configured and arranged to prevent adjustment of the separating distance between the two outward facing surfaces of the two endplates once the lock is engaged.

8. The artificial intervertebral implant of claim 7, wherein the lock is a jam-nut which is threadingly engaged with the central adjuster, the jam-nut being capable of being frictionally engaged with a surface of one of the two endplates.

9. The artificial intervertebral implant of claim 7, wherein the lock includes an elongated bar that penetrates through one of the two endplates, the bar having a tip configured to frictionally engage a surface of the central adjuster.

10. The artificial intervertebral implant of claim 7, wherein the lock includes an elongated bar having a tip configured to matingly engage into a complementary cavity.

11. The artificial intervertebral implant of claim 10, wherein the central adjuster has a series of complementary cavities, each configured to accommodate receipt of the tip of the bar separately, the cavities being arranged in such a manner that only a limited number of adjustments in the height of the implant are attained corresponding to the series.

12. The artificial intervertebral implant of claim 10, wherein the cavity and the tip of the bar each configured to matingly fit into each other.

13. The artificial intervertebral implant of claim 10, wherein the complementary cavity and the lock being in different ones of the two endplates and the central adjuster.

14. The artificial intervertebral implant of claim 13, wherein the central adjuster and the two endplates have threads that engage endplate in one direction to expand the separating distance between the outward facing surfaces of the two endplates, the lock and the complementary cavity having threads that engage each other in a direction opposite to the one direction to prevent the adjustment.

15. The artificial intervertebral implant of claim 1, wherein the articulating surface is configured to allow articulations in a manner that selected from a group consisting of axial rotation, flexion, extension, lateral bending, translation, and any combination thereof.

16. The artificial intervertebral implant of claim 15, wherein at least a portion of the articulating surface is in a shape selected from a group consisting of concave, convex, and a mixture of concave and convex.

17. The artificial intervertebral implant of claim 1, further comprising a positive stop configured and arranged to limit an extent of articulation that the articulating surface may articulate.

18. The artificial intervertebral implant of claim 17, wherein the positive stop is an integral part of one of the endplates.

19. The artificial intervertebral implant of claim 17, wherein the positive stop is a separate structure attached to one of the endplates.

20. The artificial intervertebral implant of claim 17, wherein the positive stop is arranged in such a manner to allow articulation in different direction by different extents.

21. The artificial intervertebral implant of claim 1, wherein the central element further comprises a further articulating surface that articulates with a corresponding surface of a further of the two endplates.

22. The artificial intervertebral implant of claim 21, wherein the central adjuster is comprised of at least two components, one comprising the articulating surface and the other comprising the further articulating surface, with the two components being so configured that the relative position of the two components can be adjusted to change the separating distance between the outward facing surfaces of the two endplates.

23. The artificial intervertebral implant of claim 22, wherein the components of the central adjuster are both threadingly engage by a retaining ring.

24. The artificial intervertebral implant of claim 23, wherein threading between the retaining ring and one component in one of a clockwise and counterclockwise direction and threading between the retaining ring and the further of the at least two endplates being in the other of the clockwise and counterclockwise directions.

25. The artificial intervertebral implant of claim 1, wherein the outward facing surfaces are roughened.

26. The artificial intervertebral implant of claim 1, wherein at least one of the two endplates have at least one protrusion that extends outwardly beyond the outward facing surface associated with the at least one of the two endplates.

27. The artificial intervertebral implant of claim 1, wherein a portion of the central adjuster is configured so that it is geometrically shaped to be snap-fitted into a corresponding cavity in one of the two endplates.

28. The artificial intervertebral implant of claim 27, wherein the endplate comprises at least one slot configured to widen in response to deformation imposed on the endplate to snap-fit with the central adjuster.

29. The artificial intervertebral implant of claim 27, further comprising a capture ring configured and arranged to confine the central adjuster inside of the corresponding cavity.

30. The artificial intervertebral implant of claim 1, wherein a portion of one of the two endplates is configured so that it is geometrically shaped to be snap-fitted into a corresponding cavity in the central adjuster.

31. The artificial intervertebral implant of claim 29, wherein the central adjuster comprises at least one slot configured to widen in response to deformation imposed on the central adjuster to snap-fit with the endplate.

32. The artificial intervertebral implant of claim 27, further comprising a capture ring configured and arranged to confine the endplate inside of the corresponding cavity.

33. The artificial intervertebral implant of claim 1, wherein the endplate further comprises a cavity inwardly from the outward facing surface.

34. The artificial intervertebral implant of claim 33, wherein the cavity extends through the endplate.

35. The artificial intervertebral implant of claim 33, wherein the endplate further comprises a channel that extends between an outer surface of the endplate to the cavity so as to be in fluid communication with the cavity to enable a fusion promoting material to be introduced into the cavity from the channel.

36. The artificial intervertebral implant of claim 35, wherein a structure is configured and arranged within the channel to prevent the fusion promoting material from backing out of the channel.

37. The artificial intervertebral implant of claim 35, wherein the channel is at least partially closed by a separate component.
